(19) United States
(12) Patent Application Publication
(10) Pub. No.: US 2007/0208172 A1
(43) Pub. Date: Sep. 6, 2007

(54) Process for Preparing Cephalosporin Intermediates Using Alpha-Iodo-1-Azetidineacetic Acid Esters and Trialkylphosphites

(75) Inventors: Hiromasa Morita, Chita-Shi (JP); Isao Nagakura, Aichi-Ken (JP); Timothy Norris, Gales Ferry, CT (US)

Correspondence Address: PHARMACIA & UPJOHN, 7000 Portage Road, KZO-300-104, KALAMAZOO, MI, 49001, US

(73) Assignee: Pfizer Inc.

(21) Appl. No.: 10/592,201

(22) PCT Filed: Feb. 25, 2005
(86) PCT No.: PCT/IB05/00508

§ 371 Date: Nov. 15, 2006

Related U.S. Application Data
(60) Provisional application No. 60/551,845, filed on Mar. 9, 2004.

(51) Int. Cl. C07D 501/00; C07D 205/08

(52) U.S. Cl. 540/222; 540/354

(57) ABSTRACT

This invention relates a process for preparing a compound of formula (I) 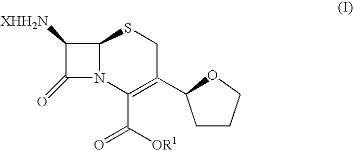
wherein R¹ is para-nitrobenzyl or allyl; X is halo; as well as its isomers.

### FIELD OF THE INVENTION

The present invention relates to the synthesis of cephalosporin intermediates for the preparation of cefovecin.

### BACKGROUND OF THE INVENTION

Cefovecin is a potent stable antibiotic targeted for companion animals. Cefovecin features a chiral tetrahydrofuran ring substituent at C₃, which is responsible for the unique activity and stability profile.

The total synthesis of Cefovecin from penicillin G consists of 15 transformations, many of which are telescoped steps. The intermediates are often variable mixtures of diastereoisomers. It is not until cephalosporin intermediates are reached that single crystalline diastereoisomers are obtained. As a result, cephalosporin intermediates were targeted as a key control gate in the synthesis of cefovecin and their synthesis is critical to the establishment of a commercial process for the production of cefovecin.

J. H. Bateson et al., The Journal of Antibiotics, 47, 253-256 (1994) provided a process of preparing cephalosporin intermediates by first converting a β-lactam to a chloro compound using thionyl chloride and then reacting the chloro compound with a trialkylphosphine to form a phosphonium salt. However, this process involves the use of standard phosphine reagents, such as triethylphosphite, tributylphosphine and triphenylphosphines, which gave poor yields of cephalosporin intermediates.

U.S. Pat. Nos. 6,077,952 and 6,001,997 as well as U.S. Patent Application Publication No. 2002/0099205 provided that the use of trimethylphosphine (TMP) provides better yield and was used successfully on large-scale. There are a number of disadvantages to the use of TMP in this process, such as high expense, highly variable yields and relatively unstable intermediates.

U.K. Patent Application No. 2,300,856 provided alternative processes for syithesizing cephalosporin intermediates. However, these processes have relatively low yields. Therefore, there is a need to develop new processes for the synthesis of cephalosporin intermediates.

### BRIEF SUMMARY OF THE INVENTION

The present invention relates to a processes of preparing a compound of formula (IVa)

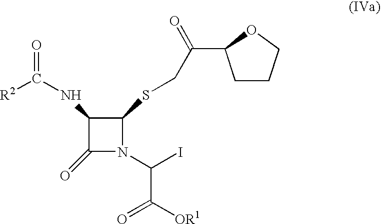

wherein R¹ is para-nitrobenzyl or allyl and R² is benzyl or substituted benzyl; comprising the step of reacting a compound of formula (V)

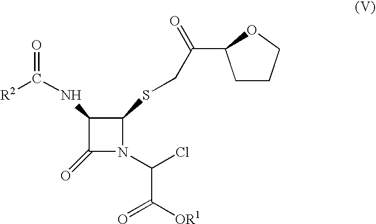

wherein R¹ and R² are as defined above; with an iodide salt to produce the compound of formula (IVa).

Suitable iodide salts include, but are not limited to sodium iodide, potassium iodide, lithium iodide, calcium iodide and ammonium iodide. Preferably the iodide salt is sodium iodide.

In preferred embodiments of the invention, R¹ is para-nitrobenzyl, R² is benzyl substituted with 1-3 substituents each independently selected from the group consisting of C₁₋₆alkyl, or halo.

Suitable chlorinating agents for conversion of the compound of formula (VI) into the compound of formula (V) include thionyl chloride and phosphorus oxychloride. Preferably, the chlorinating agent is thionyl chloride.

The present invention also relates to processes of preparing a compound of formula (III)

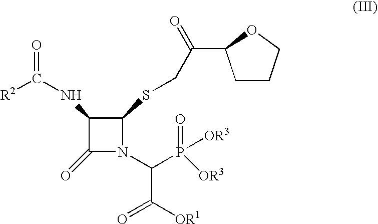

wherein R¹ is para-nitrobenzyl or allyl; R² is benzyl or substituted benzyl; and R³ is C₁₋₆alkyl; comprising the step of reacting a compound of formula (IVa)

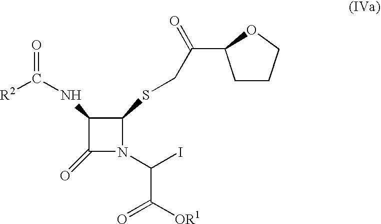

with P(OR³)₃ in a solvent; wherein R¹, R² and R³ are as defined above.

In a preferred embodiment of the invention, R¹ is para-nitrobenzyl in the process of preparing compounds of formula (III).

In another embodiment of the invention, R² is benzyl in the process of preparing compounds of formula (III).

In another embodiment of the invention, R³ is methyl and X is chloro in the process of preparing compounds of formula (III).

In another embodiment of the invention, R¹ is para-nitrobenzyl, R² is benzyl, R³ is methyl and X is chloro in the process of preparing compounds of formula (III).

In another preferred embodiment of the invention, the compound of formula (III) is heated in a solvent in the presence of LiCl and an organic soluble base to form a compound of formula (II):

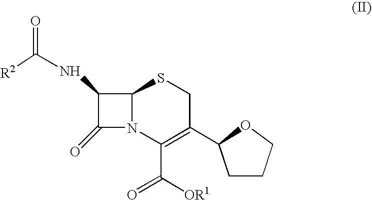

wherein R¹ is para-nitrobenzyl or allyl; and R² is benzyl or substituted benzyl; and the compound of formula (II) further reacts with R⁴—OH and PX₅ to produce the compounds of formula (I):

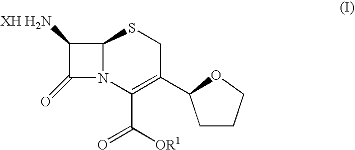

wherein R¹ is as defined above and R⁴ is C₁₋₆alkyl and X is halo.

In another preferred embodiment, R¹ is para-nitrobenzyl in the conversion of the compound of formula (III) to the compound of formula (I).

In another preferred embodiment, R² is benzyl benzyl substituted with 1-3 substituents each independently selected from the group consisting of C₁₋₆alkyl, or halo in the conversion of the compound of formula (III) to the compound of formula (I).

In another preferred embodiment, R³ is methyl in the conversion of the compound of formula (III) to the compound of formula (I).

In another preferred embodiment, R¹ is para-nitrobenzyl, R² is benzyl, R³ is methyl, X is chloro; and R⁴ is isobutyl in the conversion of the compound of formula (III) to the compound of formula (I).

In another preferred embodiment, the organic soluble base is diisopropylethylamine and the solvent is dichloromethane in the conversion of the compound of formula (III) to the compound of formula (II).

The present invention further relates to a process of preparing a compound of formula (I)

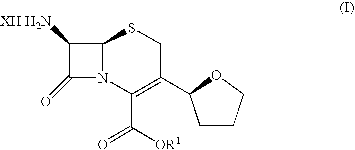

wherein R¹ is para-nitrobenzyl or allyl; and X is halo; comprising the steps of:
(1) reacting a compound of formula (V) 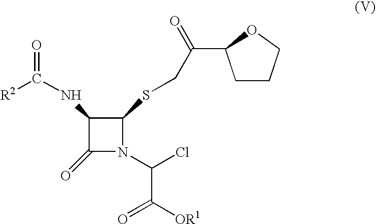
wherein R¹ is para-nitrobenzyl or allyl and R² is benzyl or substituted benzyl; with an iodide salt to produce a compound of formula (IVa) 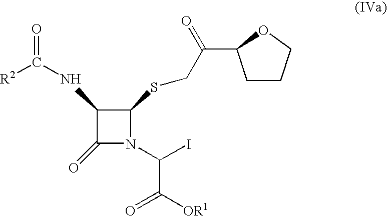
(2) reacting the compound of formula (IVa) with P(OR³)₃ in a solvent to obtain a compound of formula (III)

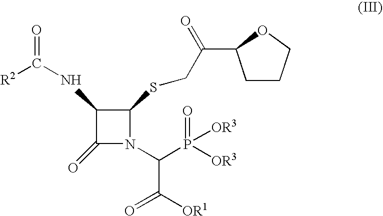

wherein R¹ and R² are as defined above; and R³ is C₁₋₆alkyl;
(3) heating the compound of formula (III) from step (2) in said solvent in the presence of LiCl and an organic soluble base to form a compound of formula (II):

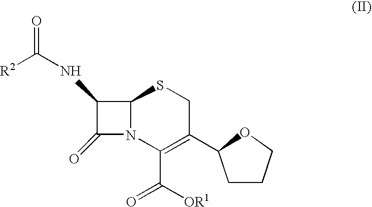

wherein R¹ is para-nitrobenzyl or allyl; and R² is benzyl or substituted benzyl; and
(4) reacting the compound of formula (II) with R⁴—OH and PX₅ to produce the compounds of formula I; wherein R⁴ is C₁₋₆alkyl and X is halo.

In a preferred embodiment, R¹ is para-nitrobenzyl in the conversion of the compound of formula (V) to the compound of formula (I).

In another preferred embodiment, R² is benzyl substituted with 1-3 substituents each independently selected from the group consisting of C₁₋₆alkyl, or halo in the conversion of the compound of formula (V) to the compound of formula (I).

In another preferred embodiment, R³ is methyl in the conversion of the compound of formula (V) to the compound of formula (I).

In another preferred embodiment, X is chloro in the conversion of the compound of formula (V) to the compound of formula (I).

In another preferred embodiment, R¹ is para-nitrobenzyl, R² is benzyl, R³ is methyl and X is chloro in the conversion of the compound of formula (V) to the compound of formula (I).

Suitable solvents for the conversion of the compound of formula (VI) to the compound of formula (III) include, but are not limited to toluene, xylene, tetrahydrofuran, dichloromethane or acetonitrile. Preferably the solvent is dichloromethane.

Suitable organic soluble bases for the conversion of the compound of formula (III) to the compound of formula (II) include, but are not limited to diisopropylethylamine (“DIPEA”), di-butylethylamine, methylpyrrolidine, ethylpyrrolidine, methylpiperidine, ethylpiperidine, ethylmorpholine, and methylmorpholine, di-cyclohexanemethylamine, di-cyclohexaneethylamine, and N,N′-dibutylurea (“DBU”).

Preferably the organic soluble base is present, during the conversion of the compound of formula (III) to the compound of formula (II), in a range from about 1 to about 2 equivalents for every mole of the compound of formula (III), preferably in a range from about 1.2 to about 1.5 equivalents.

The conversion of the compound of formula (III) into the compound of formula (II) may be conducted at a temperature of from about 0° C. to about 60° C.; preferably from about 5° C. to about 50° C., more preferably from about 5° C. to about 30° C. The aforesaid conversion may be conducted for a period from about 1 hour to about 16 hours, preferably from about 4 hours to about 10 hours.

As used herein the term “halo” includes chloro, bromo, iodo and fluoro.

Examples of substituted benzyl include, but are not limited to benzyl substituted with 1-3 substituents each independently selected from the group consisting of C₁₋₆alkyl, or halo.

The present invention further relates to a compound of formula (IV) 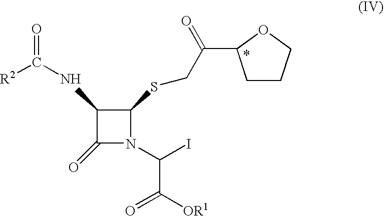
wherein R¹ is para-nitrobenzyl; R² is benzyl; wherein * represents a chiral center which represent an absolute configuration of (R) or (S); wherein said compound contains (R) and (S) isomers at a ratio between 0:1 and 1:0.

The present invention further relates to a compound of the formulas (IVa) or (IVb):

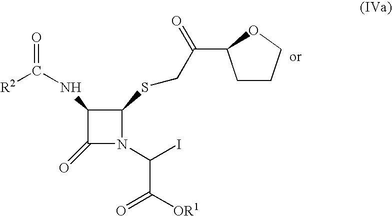

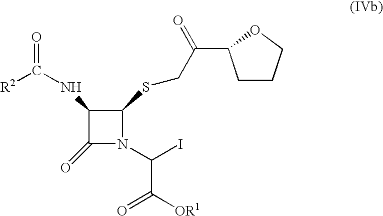

wherein R¹ is para-nitrobenzyl; R² is benzyl;

Various patents and publications were cited throughout the present application. The contents of these patents and publications and the contents of documents cited in these patents and publications are hereby incorporated herein by reference to the extent permitted.

### DETAILED DESCRIPTION OF THE INVENTION

The process of the present invention and the preparation of the compounds of the present invention are illustrated in the following reaction scheme. Except where otherwise indicated, in the reaction scheme and discussion that follow, substituents wherein R¹, R², R³, R⁴, and X are as defined above.

Compounds of formula I can be synthesized by the following scheme:

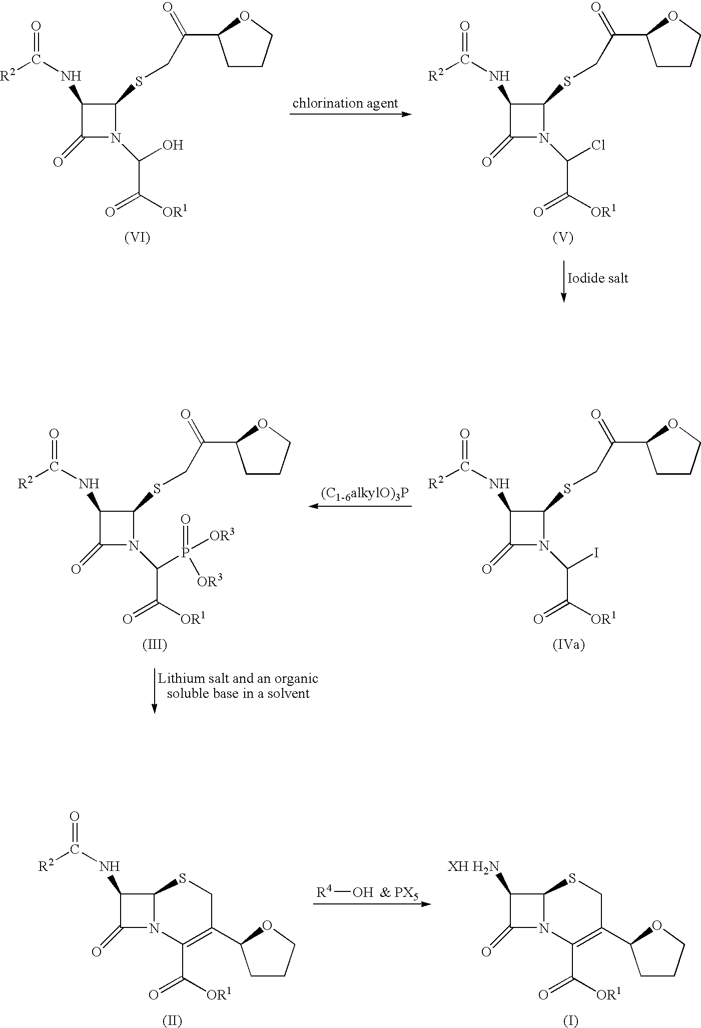

wherein R¹ is para-nitrobenzyl or allyl; R² is benzyl or substituted benzyl; R³ is C₁₋₆alkyl; X is chloro and R⁴ is isobutyl.

The preparation of compounds of formula (VI) was described in U.S. Patent Application Publication No. 2002/0099205 and the contents of which are incorporated herein by reference.

Preparation of the Chloride

The conversion of compounds of formula (VI) into compounds of formula (V) is typically conducted by chlorinating the above compounds of formula (VI) using a chlorination agent, such as thionyl chloride in an organic solvent, such as toluene, xylene, tetrahydrofuran, dichloromethane and acetonitrile with 2-picoline. This conversion gives compounds of formula V in near quantitative yield. The optimum conditions for the chlorinating agent charge was found to be about 1.1 equivalents, based on the initial charge of compounds of formula (VI). Lower charges of chlorinating agent gave incomplete conversion to compounds of formula V.

To avoid the formation of side-products, this reaction must be conducted at low temperature. However, the solution of the compounds of formula (VI) and 2-picolene in dichloromethane produce some precipitation when it was cooled from ambient temperature to −20° C. Addition of thionyl chloride to this suspension at −20° C. gave a higher amount of unreacted starting material, which could not be chlorinated by addition of excess thionyl chloride. Therefore, a portion of the total thionyl chloride charge (10%) was added before precipitation commenced, at −15° C. The solution was then cooled −20° C. and the remaining thionyl chloride was added slowly at or below this temperature. The product is significantly more soluble in dichloromethane and no precipitation was observed using this procedure.

Compounds of formula (VI) and formula (V) are diastereomeric mixtures, of the hydroxy and chloro epimers, respectively. Thin liquid chromatography (“TLC”) of the chlorination reaction mixture showed clean conversion of compounds of formula (VI) to compounds of formula V with a small amount of unreacted compounds of formula (VI) and baseline material. None of the diastereomers were resolved.

The four possible diastereomers were resolved using reversed-phase HPLC. However, the RP-HPLC result was not consistent with the TLC. It indicated that the reaction mixture contained approximately 50% of compounds of formula (V) which is mainly one epimer, and 50% of compounds of formula (VI), also mainly one epimer. Normal-phase HPLC was consistent with the TLC, and showed the reaction conditions used gave greater than 90% conversion to compounds of formula (V), with 3-10% of compounds of formula (VI) remaining. These observations suggested that one epimer of the product hydrolyses rapidly in the RP-HPLC, whereas the other is relatively stable.

Fortunately, although the reaction was quenched into saturated brine and dried over magnesium sulfate before proceeding with the phosphonate formation, the work-up procedure did not cause any significant hydrolysis of compounds of formula (V).

Preparation of the Phosphonate

The conversion of compounds of formula V into compounds of formula (III) is typically conducted by reacting an alkyl halide with a trialkylphosphite (Arbuzov reaction) or an alkali metal derivative of the dialkyl phosphate (Michaelis reaction). The Arbuzov reaction offers simpler reaction conditions (J. Boutagy & R. Thomas, Chem. Rev. 1, 87-99 (1974) and was developed for the preparation of compounds of formula (III).

Trimethylphosphite, triethylphosphite and tributylphosphite do not react with the chloro compounds of formula (IVa) and the chloride was exchanged for iodide by reaction with an iodide salt, such as sodium iodide (Finkelstein reaction). This was initially performed by adding sodium iodide to the reaction solution containing compounds of formula (V), after the aqueous work-up and drying.

Due to the low solubility of sodium iodide in dichloromethane, this procedure gave inconsistent yields and purities of compounds of formula (IVa). Dosing trace amount of water into the reaction mixture increases the solubility of sodium iodide. However, when the dichloromethane contained sufficient water to dissolve enough sodium iodide to allow the reaction to proceed, significant hydrolysis occurred.

Alternative solvents for the Finkelstein reaction were tried. The use of acetone (and other ketone containing solvents, e.g. methyl ethyl ketone) was avoided due to its potential to compete with the internal ketone during the cyclization of compounds of formula (III). Acetonitrile was found to be a good solvent for the halide exchange in terms of both product yield and quality. Some degradation occurred if the reaction solution containing compounds of formula (V) was evaporated to dryness and the residue dissolved in acetonitrile. However, the halide exchange reaction could be performed by drying and then concentration of the reaction solution containing compounds of formula (V) after work-up and drying, and then dilution with acetonitrile, followed by addition of the sodium iodide.

The charge of iodide salt is critical to the yield of compounds of formula (IVa). Insufficient iodide salt results in a yield reduction through incomplete reaction of compounds of formula (V). Excess iodide salt causes decomposition of compounds of formula (IVa) by reacting with these compounds. About 1.05 mole equivalents of iodide salt, based on the initial charge of compounds of formula (VI), was used in converting compounds of formula (V) to compounds of formula (VI).

Compounds of formula (V) are converted to compounds of formula (IVa) within minutes of the addition of iodide salt. Our experience with the Wittig synthesis suggested that the use of the least sterically hindered trialkylphosphite for the Arbuzov reaction with compounds of formula (IVa) would be advantageous. Trimethylphosphite (“TMPT”) gave good conversion of compounds of formula (IVa) to the corresponding compounds of formula (III).

Compounds of formula (III) were prepared by adding TMPT to the solution containing compounds of formula (IVa). The reaction of TMPT with compounds of formula (IVa) is exothermic and requires careful temperature control as higher temperature increases the production of the phosphate impurity (see FIG. 1). The exotherm was controlled by cooling the solution containing compounds of formula IV to below 5° C. before the slow addition of TMPT in dichloromethane solution.

The optimum TMPT charge was about 1.45 mole equivalents, based on the initial charge of compounds of formula (VI). Lower charges of TMPT gave incomplete conversion of compounds of formula (V) to compounds of formula (IVa) and higher charges gave rises to problems later in the synthesis (in the PX₅ deprotection) due to the telescopic design of the process.

Compounds of formula (III) were fully formed after reaction for one and half hours at room temperature. An HPLC solution assay for compounds of formula (III) were developed, which showed a yield of 75% from compounds of formula (VI). It was important to determine the content of compounds of formula (III) in the reaction mixture so that subsequent reagent charges could be based on the result.

Cyclization of the Phosphonate

The cephem 6-membered ring cyclization is performed by adding a lithium salt, such as lithium chloride, lithium fluoride and lithium bromide, and an organic soluble base, such as DIPEA to the reaction solution containing compounds of formula (III). The reaction proceeds via formation of a (stabilized) phosphonate anion, which cyclizes internally to give the product, compounds of formula (III), which contains the fully formed bicyclic cephalosporin nucleus. At least two mole equivalents of lithium salt were required for successful cyclization. Excess lithium salt had no deleterious effect.

A number of bases were investigated and diisopropylethylamine, DIPEA, was found to be very effective in the cyclization reaction. Other soluble bases, such as di-butylethylamine, methylpyrrolidine, ethylpyrrolidine, methylpiperidine, ethylpiperidine, ethylmorpholine, and methylmorpholine, di-cyclohexanemethylamine, di-cyclohexaneethylamine, and DBU can also be used. However, the use of bases that are weaker than DIPEA were unsuccessful, probably because they are not able to deprotonate the phosphonate.

Without intending to be bound by any particular theory of operation, it is believed that a major difference between the phosphite and phosphine routes is the potential for Δ2-3 double bond isomerisation during the cyclisation step in the phosphite method. Isomerisation of the double-bond in the cephalosporin ring is promoted by the base. In the Wittig synthesis the ylid is formed by treatment of the phosphonium salt in dichloromethane with aqueous sodium bicarbonate. The organic phase is separated and allowed to cyclize at ambient temperature, which takes up to 16 hours. Since DIPEA is a stronger base than bicarbonate, and it is difficult to remove from the reaction until after the cyclization, the DIPEA charge is critical. The amount of isomerization is directly related to the DIPEA charge. An optimal amount of DIPEA is in the range of 1.20 to 1.50 equivalents, based on the mole amount of the phosphonate.

This ensures complete reaction and minimizes the formation of the double-bond isomer. The amount of phosphonate in the reaction solution was determined by HPLC assay and the DIPEA and lithium chloride charges were based on the result.

After addition of DIPEA and lithium chloride, the solution was stirred at ambient temperature to effect the cyclization, which required more than 16 hours to go to completion. The use of a higher reaction temperature and/or significantly longer reaction times led to an increase in side products and lower yield.

It was found that residual water in the cyclization reaction mixture resulted in the formation of impurities and lower yields. Therefore, the solution of phosphonate was dried over magnesium sulfate before addition of the sodium iodide, lithium chloride and DIPEA.

Deprotection of the Compounds of Formula II

The conversion of the compounds of formula (II) to compounds of formula (I) involves the deprotection of the amino groups in compounds of formula (II). The deprotection uses the standard conditions in cephalosporin chemistry, phosphorous pentahalide, picoline, then isobutanol. Compounds of formulas (VI) and (III) require the presence of acetonitrile in the reaction solution. However, it was necessary to remove the acetonitrile prior to proceeding with the final deprotection reaction of compounds of formula (II) because acetonitrile reacts with the phosphorous pentahalide. It increases the solubility of the compounds of formula (I) in the reaction mixture and results in a lower yield.

There were two possible times to remove the acetontrile, after formation of compounds of formula (III), or after formation of compounds of formula (II). There are two methods to remove acetontrile, distillation and phase-extraction. It was found that removal of the acetonitrile after formation of compounds of formula (VI) by distillation affected the product impurity profile and yield of compounds of formula (II). Likewise, removal of acetonitrile by extraction of the reaction mixture containing compounds of formula (IVa), leads to emulsions, low yield and recovery and issues with the reaction water content when proceeding to the subsequent cyclisation step. Thus the only step available to remove the acetonitrile was immediately prior to the deprotection of compounds of formula (II). The reaction mixture was extracted with acid solution to remove the DIPEA salts, followed by brine, and this removed some of the acetonitrile. The reaction mixture was then distilled twice, to ensure complete removal of acetonitrile.

There are two major issues with this conversion. One is the presence of residual water and the other is the control of reaction temperature/exotherms. These issues are common to both the phosphite and phosphine routes. The water content needs to be low, and this is achieved through the distillation procedure to remove the acetonitrile. In addition, it has been found that the deprotection reaction works consistently well on compounds of formula (II) which have been isolated and purified, but is variable when using compounds of formula (II) produced via this telescoped series of reactions from compounds of formula (VI). This suggests that some other component(s) in the reaction solution have a detrimental effect on the deprotection reaction.

Dimethylphosphate (“DMP”) is a byproduct of the cyclization reaction. DMP and the excess TMPT were shown to be negatively effect the deprotection and are not removed by the aqueous work-up of compounds of formula (II). Based on this observation, excess TMPT charge used in the preparation of compounds of formula (III) from compounds of formula (IVa) was kept a minimum, which was found to be 1.45. There is some data to suggest that 10A molecular sieves remove the phosphorous compounds from the reaction mixture.

The following Examples illustrate the preparation processes of the present invention. NMR data are reported in parts per million (ppm) and are referenced to the deuterium lock signal from the sample solvent (deuteriochloroform unless otherwise specified).

Further, any range of numbers recited in the specification or paragraphs hereinafter describing or claiming various aspects of the invention, such as that representing a particular set of properties, units of measure, conditions, physical states or percentages, is intended to literally incorporate expressly herein by reference or otherwise, any number falling within such range, including any subset of numbers or ranges subsumed within any range so recited. The term “about” when used as a modifier for, or in conjunction with, a variable, is intended to convey that the numbers and ranges disclosed herein are flexible and that practice of the present invention by those skilled in the art using temperatures, concentrations, amounts, contents, carbon numbers, and properties that are outside of the range or different from a single value, will achieve the desired result.

### EXAMPLE 1

### Preparation of (3R,4R)-(4-nitrophenzyl)methyl ester-α-iodo-2-oxo-4-[[2-oxo-2-[(1S)-(tetrahydro-2-furanyl]-ethyl]thio]-3-[(phenylacetyl)amino]-1-azetidine acetic acid

The four-membered ring compound (3R,4R)-(4-nitrophenzyl)methyl ester-α-hydroxy-2-oxo-4-[[2-oxo-2-[(1S)-(tetrahydro-2-furanyl]-ethyl]thio]-3-[(phenylacetyl)amino]-1-azetidine acetic acid is a mixture of diastereomeric alcohols in the ratio 8:2. The absolute stereochemistry of the pair is not known at the alcohol carbon. 51.19 g of the compound (80% potency, 73.4 mmol) was dissolved in 750 mL dichloromethane. 2-Picoline (11.8 mL) (119.5 mmol, 1.63 equivalent) was added and the solution was cooled to −15° C. Thionyl chloride (7.6 mL) (104.19 mmol, 1.42 equivalent) was added in one portion (over approximately 3 minutes). The reaction was stirred for 1 hour below −20° C. It was washed with 2×250 mL 20% brine solution and dried over 40 g of magnesium sulfate for 10 minutes at ambient temperature. The desiccant was filtered off and washed with 100 mL dichloromethane. The filtrate was concentrated to 150 mL on a rotary evaporator at less than 35° C. Acetonitrile (150 mL) was added and the solution further concentrated to 200 mL at less than 35° C.

The solution was cooled to less than 5° C. Sodium iodide (11.59 g) (119.5 mmol, 1.05 equivalents to the starting compound) was charged to the solution to form (3R,4R)-(4-nitrophenzyl)methyl ester-(α-iodo-2-oxo-4-[[2-oxo-2-[(1S)-(tetrahydro-2-furanyl]-ethyl]thio]-3-[(phenylacetyl)amino]-1-azetidine acetic acid, which can exist in the form of (S)-THF isomer or the (R)-THF isomer or their mixture. Moreover, both the (S)-THF isomer and the (R)-THF isomer can exist in the form of a mixture of iodo stereomers which consist of the (S)-iodo isomer and the (R)-iodo isomer. The (S)-THF isomer is used for the preparation of the cephalosporin intermediate and cefovecin. The (R)-THF isomer is present as an impurity in all the process intermediates along the way of preparing cefovecin and in the final product. However, the (R)-THF isomer of cefovecin sodium was shown to be a potent anti-microbial in its own right in the initial screening tests.

NMR data collected on the iodo compound mixture, predominantly the S-series with traces of the R series present: δ (400 MHz, CDCl₃): 8.43 (m, 2H, PNB-H2,6), 7.54 (m, 2H, PNB-H3,5), 7.20-7.40 (m, 5H, Bnz-H), 6.5-6.7 (m, 1H, NH), 5.2-5.45 (m, 4H, PNB—CH₂, CH—OH & CH—NH), 5.07 (d, 1H, J=4.8 Hz, CH—S¹), 4.2-4.5 (m, 1H, THF-H2), 3.83 (m, 2H, THF-H5), 3.3-3.7 (m, 4H, S—CH₂ & Bnz-CH₂), 2.1-2.2 (m, 1H, THF-H3), 1.7-1.95 (m, 3H, THF-H3 & H4).

MS data: 690.0382 (M+Na)⁺.

HPLC data: 42.2% of the two epimers of the above Iodo compounds (Rt 12.6 & 14.5 min.), 8.4% of the two epimers of Chloro analogs of the iodo compounds (Rt 12.2 & 14.1 min), 11.4% of the two epimers of (3R,4R)-(4-nitrophenzyl)methyl ester-α-hydroxy-2-oxo-4-[[2-oxo-2-[(1S)-(tetrahydro-2-furanyl]-ethyl]thio]-3-[(phenylacetyl)amino]-1-azetidine acetic acid (Rt 19.6 & 20.5 min).

### EXAMPLE 2

### Preparation of Cephalosporin Intermediate

The Addition of sodium iodide in example 1 was followed by trimethylphosphite (TMPT) (12.6 mL, 106.8 mmol, 1.45 equivalents relative to the starting compound) dissolved in dichloromethane (10 mL), added dropwise over 10 minutes. The temperature was maintained at or below 5° C. during the addition. No exotherm was observed on this scale. The solution was allowed to warm to room temperature over 1.5 hours. The-phosphonate content was determined by HPLC assay (36.49 g, 56.2 mmol). This corresponds to a yield of 76.5% for the two steps. Dichloromethane (500 ml) was added (total volume approximately 700 mL). Activated carbon (17 g) and magnesium sulfate (20.1 g) were added and the mixture stirred for 10 minutes. The mixture was clarified by filtration through a bed of celite and the celite washed with dichloromethane (150 mL). The phosphonate content was determined by HPLC assay (36.5 g, 56.1 mmol). Lithium chloride (5.11 g) (120.5 mol, 2.15 equivalents of the phosphonate) and DIPEA (12.6 mL) (72.3 mmol, 1.29 equivalents of the phosphonate) were added. The solution was stirred at ambient temperature for 16 hours. The reaction solution was successively washed with 400 mL of 1% aqueous hydrochloric acid and 2×400 mL of 20% brine solution. The organic phase was dried with powered 4A molecular sieves. (22.3 g) and celite (20.3 g). The desiccant was decanted off through a plug of silica G (43 g) and washed with 200 mL dichloromethane. The solution was concentrated to a thick oil on a rotary evaporator at less than 35° C. and dichloromethane (350 mL) added. This solution was then re-concentrated to a thick oil on a rotary evaporator at less than 35° C. and dichloromethane (350 mL) was added. The water content was determined to be 140 ppm. The content of the cyclization product was determined by HPLC assay, as 25.76 g (49.2 mmol, 67.0% yield from 3, 87.6% for the cyclization).

The solution was cooled to −55° C. and phosphorous pentachloride (30.4 g) (147.4 mmol, 3.0 equivalents of the cyclization product) was charged. After 5 minutes, 2-picoline (29 mL) (293.6 mmol, 6.0 equivalents of the cyclization product) was added while the temperature was maintained at below −40° C. An exotherm was observed. The solution was stirred for 1 hour below −20° C. At this stage the reaction was a thick slurry. It was cooled to below −50° C. and isobutanol (205 mL) (2.02 mol) was charged. This caused the reaction to warm to −30° C. The solution was allowed to warm to ambient temperature and after stirring for 1 hour, a seed crystal of cephalosporin intermediate was added. The solution was stirred for 16 hours in a closed system to avoid evaporation of the dichloromethane. The solid was collected by filtration. The solid was washed with 2×100 ml of dichloromethane. The solid was dried to constant weight at 40° C. under high vacuum to give cephalosporin intermediate (18.4 g) (41.64 mmol, 56.7% yield from (3R,4R)-(4-nitrophenzyl)methyl ester-α-hydroxy-2-oxo-4-[[2-oxo-2-[(1S)-(tetrahydro-2-furanyl]-ethyl]thio]-3-[(phenylacetyl)amino]-1-azetidine acetic acid, 84.6% yield from the cyclization product).

Three additional lots of cephalosporin intermediate were prepared in very similar yields (50-55%) on 50 g scale. The overall yield is comparable with the best achievable with the phosphine method.

Batches of the cephalosporin intermediate prepared using this method were found to have a similar impurity profile to that produced by the original phosphine (Wittig) method. They have been used to prepare Cefovecin that met all of the current test specifications for drug substance release.

### EXAMPLE 3

### Preparation and Identification of the Phosphonate

51.8 g (3R,4R)-(4-nitrophenzyl)methyl ester-α-hydroxy-2-oxo-4-[[2-oxo-2-[(1S)-(tetrahydro-2-furanyl]-ethyl]thio]-3-[(phenylacetyl)amino]-1-azetidine acetic acid (RD2424, 80%, 73.4 mmol) was dissolved in 750 mL dichloromethane. 12 mL 2-picoline (121.5 mmol, 1.63 equivalent to, ALAT) were added and the solution was cooled to −15° C. Added 7.5 mL of thionyl chloride (102.82 mmol, 1.38 equivalent to ALAT). The reaction was stirred for 1 hour at below −20° C. It was washed with 2×250 mL of 20% brine and dried over 40 g of magnesium sulfate, for 10 minutes at ambient temperature. The desiccant was filtered off and washed with 100 mL of dichloromethane. The filtrate was concentrated to 100 mL on a rotary evaporator at less than 35° C. 150 mL of acetonitrile was added and the solution further concentrated to 200 mL on a rotary evaporator at less than 35° C. The solution was cooled to less than 4° C. Charged 11.6 g (77.4 mmol, 1.04 equivalent to (3R,4R)-(4-nitrophenzyl)methyl ester-α-hydroxy-2-oxo-4-[[2-oxo-2-[(1S)-(tetrahydro-2-furanyl]-ethyl]thio]-3-[(phenylacetyl)amino]-1-azetidine acetic acid) of sodium iodide followed by the addition of trimethylphosphite (110.22 mmol, 1.48 equivalents to (3R,4R)-(4-nitrophenzyl)methyl ester-α-hydroxy-2-oxo-4-[[2-oxo-2-[(1S)-(tetrahydro-2-furanyl]-ethyl]thio]-3-[(phenylacetyl)amino]-1-azetidine acetic acid) dissolved in dichloromethane (10 mL) added dropwise over 15 minutes. The temperature was maintained at or below 4° C. during the addition and no exotherm was observed. The solution was stirred for 1.5 hours. Dichloromethane (500 ml) was added such that the total volume was ˜700 mL. Activated carbon (17 g), 13× molecular sieves (40.00 g) and magnesium sulfate (20.1 g) were added and the solution was stirred for 10 minutes. It was filtered through a bed of celite and washed with dichloromethane (100 mL). The filtrate was concentrated on a rotary evaporator at less than 35° C. to a thick oil. This was triturated with diethyl ether (2×500 mL, the second wash was stored at 4° C. for 16 hours prior to decantation) and the semi-solid dried under vacuum to give a yellow solid (51.89 g, HPLC 60.9%, 65.4% yield).

IR (KBr disc): 3300 sh, 3281 s, 2958 s, 1779 s, 1678 s, 1607 m, 1524 s, 1454 m, 1349 s, 1261 s, 1035 s, 850 m, 739 m, 697 m cm⁻.

NMR (¹H 400 MHz, CDCl₃): 1.88 (m, 3H), 2.12 (m, 1H), 3.37-3.54 (2×dd, 2H), 3.64, (s, 2H), 3.75-3.80 (m, 6H), 3.87 (m, 2H), 3.90 (m, 1H), 4.95 (dd, 1H (J¹_{HP}=24.8 Hz)), 5.15-5.30 (dd, 0.5H (J=4.7, 1 Hz)), 5.30 (m, 2.5H), 5.46 (m (2×ddd) 1H), 6.36 & 6.46 (2×d, 1H), 7.27-7.28 (m, 5H), 7.55 (d, 2H), 8.21 (m, 2H) ppm

### EXAMPLE 4

### Cyclization of the Phosphonate

11.31 g of the phosphonate from Example 4 was dissolved in a mixture of dichloromethane (140 ml) and acetonitrile (30 ml). To this 1.33 g of LiCl (31.38 mmol) and 3.30 mL of DIPEA (18.95 mmol) were added. The solution was stirred at ambient temperature for 16 hours. The reaction solution was successively washed with 80 mL of 1% HCl and 80 mL of 20% brine. The organic phase was dried with powdered 4A molecular sieves (4.20 g), 13× molecular sieves (4.26 g) and celite (4.11 g). The desiccant was decanted off through a plug of silica (30 g) and washed with 150 mL dichloromethane. The solution was concentrated on a rotary evaporator at less than 35° C. to give a thick oil. This oil was triturated with diethyl ether (2×100 ml), and the semi-solid dried under vacuum to give a golden yellow solid (2.78 g, HPLC 87.9%, 44% yield).

IR (KBr disc): 3276 s, 3.029 m, 2949 s, 2872 m, 1783 s, 1725 s, 1666 s, 1630 s, 1610 s, 1520 m, 1454 m, 1345 s, 1219 s, 1103 s, 1053 s, 926 m, 852 s, 768 m, 737 s, 700 m, cm⁻.

NMR (¹H 400 MHz): 1.55 (m, 1H), 1.9 (m, 2H), 2.35 (m 1H), 3.25 (d, 1H SCH₂), 3.65 (d, 1H SCH₂), 3.6 (d, 2H PhCH₂CO), 3.8-3.9 (m, 2H), 4.9 (m, 1H), 4.95 (d, 1H), 5.25 (dd, 2H NO₂PhCH₂O), 5.8 (dd, 1H), 6.1 (d, 1H, NH), 7.23-7.35 (m, 5H), 7.55 (d, 2H), 8.2 (d, 2H).

### EXAMPLE 5

### Preparation of the Compound of Formula (IVb)

A compound of the formula (Vb):

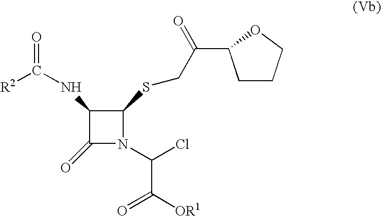

are converted to a compound of formula (IVb) with the addition of an iodide salt; wherein R¹ is para-nitrobenzyl; R² is benzyl.

While the invention has been described and illustrated with reference to certain particular embodiments thereof, those skilled in the art will appreciate that various adaptations, changes, modifications, substitutions, deletions, or additions of procedures and protocols may be made without departing from the spirit and scope of the invention. It is intended, therefore, that the invention be defined by the scope of the claims that follow and that such claims be interpreted as broadly as is reasonable.

1-18. (canceled)

19. A process of preparing a compound of formula (Iva) 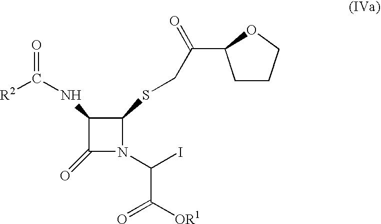
wherein R¹ is para-nitrobenzyl or allyl and R² is benzyl or substituted benzyl;
comprising the step of reacting a compound of formula (V) 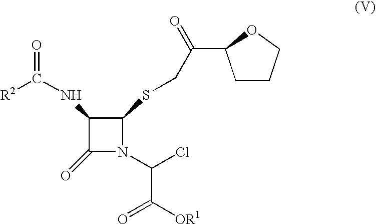
wherein R¹ and R² are as defined above;
with an iodide salt to produce the compound of formula (Iva).

20. The process of claim 19, wherein R¹ is para-nitrobenzyl.

21. The process of claim 19, wherein R² is benzyl substituted with 1-3 substituents each independently selected from the group consisting of C₁₋₆alkyl, or halo.

22. A process of preparing a compound of formula (III) 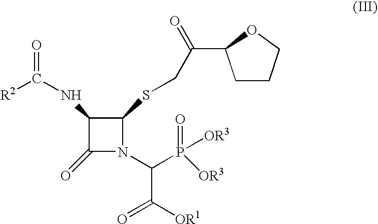
wherein R¹ is para-nitrobenzyl or allyl; R² is benzyl or substituted benzyl; and R³ is C₁₋₆alkyl;
comprising the step of reacting a compound of formula (IVa) 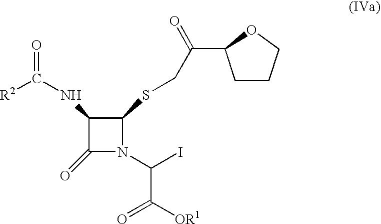
with P(OR³)₃ in a solvent; wherein R¹, R² and R³ are as defined above.

23. The process of claim 22, wherein R¹ is para-nitrobenzyl.

24. The process of claim 22, wherein R is R² is b substituted with 1-3 substituents each independently selected from the group consisting of C₁₋₆alkyl, or halo.

25. The process of claim 22, wherein R³ is methyl.

26. The process of preparing a compound of formula I 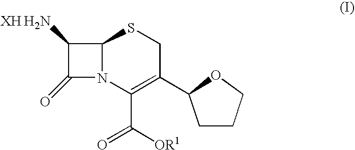
wherein R¹ is as defined above and R⁴ is C₁₋₆alkyl and X is halo comprising the process of claim 22 and then further comprising the steps of
(1) heating said compound of formula (III) in a solvent in the presence of LiCl and an organic soluble base to form a compound of formula (II) 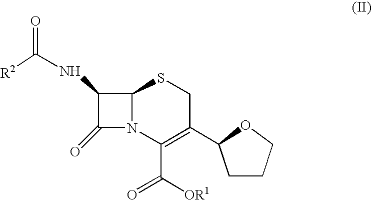
wherein R¹ is para-nitrobenzyl or allyl; and R² is benzyl or substituted benzyl; and
(2) reacting the compound of formula (H) with R⁴—OH and PX₅ to produce the compounds of formula (I);

27. The process of claim 26, wherein R¹ is para-nitrobenzyl, R² is benzyl, R³ is methyl, X is chloro; and R⁴ is isobutyl.

28. The process of claim 26, wherein said organic soluble base is diisopropylethylamine and said solvent is dichloromethane.

29. A process of preparing a compound of formula (I) 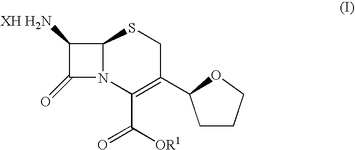
wherein R¹ is para-nitrobenzyl or allyl; X is halo;
comprising the steps of:
(1) reacting the compound of formula (V) 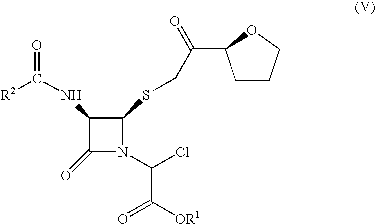
wherein R¹ is para-nitrobenzyl or allyl and R² is benzyl or substituted benzyl;
with an iodide salt to produce the compound of formula (IVa) 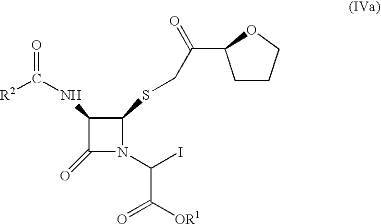
(2) reacting a compound of formula (IVa) with P(OR³)₃ in a solvent to obtain a compound of formula (III) 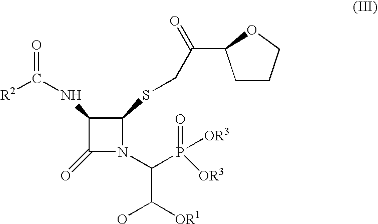
wherein R¹ and R² are as defined above; and R³ is C₁₋₆alkyl;
(3) heating the compound of formula III from step (2) in said solvent in the presence of LiCl and an organic soluble base to form a compound of formula (II) 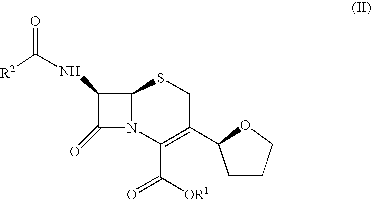
wherein
R¹ is para-nitrobenzyl or allyl;
R² is benzyl or substituted benzyl; and
(4) reacting the compound of formula (III) with R⁴—OH and PX₅ to produce the compounds of formula I; wherein R⁴ is C₁₋₆alkyl and X is halo.

30. The process of claim 29, wherein R¹ is para-nitrobenzyl.

31. The process of claim 29, wherein R² is benzyl substituted with 1-3 substituents each independently selected from the group consisting of C₁₋₆alkyl, or halo.

32. The process of claim 29, wherein R³ is methyl.

33. The process of claim 29, wherein X is chloro.

34. The process of claim 29, wherein R¹ is para-nitrobenzyl, R² is benzyl, R³ is methyl and X is chloro.

35. A compound of the formula (IV) 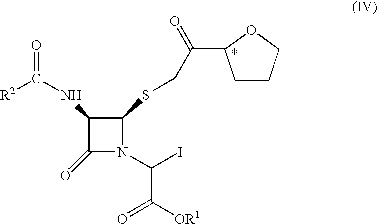
wherein R¹ is para-nitrobenzyl; R² is benzyl; wherein * represents a chiral center which represent an absolute configuration of (R) or (S); wherein said compound contains (R) and (S) isomers at a ratio between 0:1 and 1:0.

36. A compound of the formulas (IVa) or (IVb): 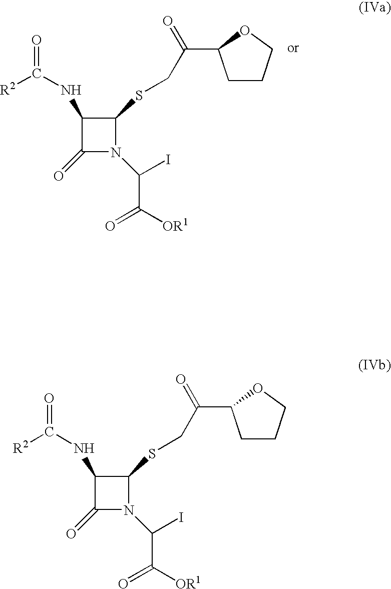
wherein R¹ is para-nitrobenzyl and
R² is benzyl.
